(19) United States
(12) Patent Application Publication
(10) Pub. No.: US 2021/0299370 A1
(43) Pub. Date: Sep. 30, 2021

(54) ARTIFICIAL RESPIRATION

(71) Applicant: Pankaj Merchia, Boca Raton, FL (US)

(72) Inventor: Pankaj Merchia, Boca Raton, FL (US)

(21) Appl. No.: 17/220,922

(22) Filed: Apr. 1, 2021

Related U.S. Application Data
(63) Continuation-in-part of application No. 17/216,667, filed on Mar. 29, 2021.
(60) Provisional application No. 63/003,884, filed on Apr. 1, 2020.
(60) Provisional application No. 63/005,196, filed on Apr. 3, 2020.
(60) Provisional application No. 63/015,707, filed on Apr. 27, 2020.
(60) Provisional application No. 62/704,392, filed on May 8, 2020.
(60) Provisional application No. 62/704,689, filed on May 22, 2020.
(60) Provisional application No. 63/001,250, filed on Mar. 28, 2020.
(60) Provisional application No. 63/003,884, filed on Apr. 1, 2020.

(51) Int. Cl. A61M 16/00; A61M 16/06; A61M 16/20

(52) U.S. Cl. CPC A61M 16/009; A61M 16/205; A61M 16/204; A61M 16/06

(57) ABSTRACT

The present invention is directed to filtered respiration that can occur according to multiple facets. The present invention includes a Continuous Positive Airway Pressure (“CPAP”) system, an adapter, and a CPAP interface. The system may be wearable and include advantageously adjustable exhausts.

### RELATED APPLICATIONS

This application claims priority under 35 U.S.C. § 120 from U.S. non-provisional patent application Ser. No. 17/216,667 titled Filtered Respiration filed on Mar. 29, 2021, which in turn claims priority under 35 U.S.C. § 119 from U.S. Provisional Patent Application No. 63/001,250 filed Mar. 28, 2020, as well as priority from U.S. Provisional Patent Application No. 63/003,884 filed Apr. 1, 2020, as well as priority from U.S. Provisional Patent Application No. 63/005,196 filed Apr. 3, 2020, as well as U.S. Provisional Patent Application No. 63/015,707 filed Apr. 27, 2020, as well as U.S. Provisional Patent Application No. 62/704,392 filed May 8, 2020, as well as U.S. Provisional Patent Application No. 62/704,689 filed May 22, 2020. This application claims priority under 35 U.S.C. § 119 from U.S. Provisional Patent Application No. 63/003,884 filed Apr. 1, 2020

### FIELD OF THE INVENTION

The present invention relates to the fields of artificial respiration and more specifically to the field of powered personal protective equipment and mechanical ventilation.

### BACKGROUND

COVID-19 is an infectious and lethal viral illness. COVID-19 is spread via respiratory methods (e.g. coughing, sneezing). The COVID-19 pandemic has resulted in a shortage of adequate masks to protect the healthy. Masks that seal well, filter inhaled air, and are tolerable to wear for long periods. COVID-19 pandemic has resulted in a shortage of adequate masks to prevent the spread of infection. Masks that seal well, filter exhaled air, and are tolerable to wear for long periods. The COVID-19 pandemic has resulted in a shortage of ventilator devices. Devices that blow air into patient's lungs to help them inhale and maintain lung volume if they develop acute respiratory distress syndrome or pneumonia.

Obstructive Sleep Apnea (“OSA”) is a common condition where the airway collapses when an individual is sleeping; partial collapse causes snoring. OSA affects about 22% of Men and 17% of women or about 1 in 5 persons (J Thorac Dis. 2015 August; 7 (8): 1311-1322). Thus OSA affects about 60 million people in the US. CPAP machines are used by millions of people to treat their obstructive sleep apnea. CPAP machines are currently used in the following system configuration as shown in FIG. 1. A CPAP machine motivator 120a blows room air into tubing 130 pressurizing the air. Tubing carries pressurized air from the CPAP motivator 120a to a patient mask, or other interface 140. A Patient mask covers patient nose and sometimes mouth as well. An exhaust vent on the mask allows exhaled air to be released into room. When a patient exhales, most of its exhaled air goes into the tubing and mask. This exhaled air is then exhausted into the room through the vent on the mask. Limitations to CPAP systems make their use by infected patients potentially unsafe because CPAP machines disperse exhaled air with pressure resulting in a patient's exhaled air being spread to others in their vicinity (e.g. healthcare workers, family members) and onto surfaces in their vicinity more than it would be spread with normal exhalation. Thus, use of CPAP by patients with an infection (e.g. COVID-19) presents an increased risk of spreading infection. Spread of infection is particularly dangerous in hospital settings.

The COVID-19 pandemic has resulted in a shortage of ventilators. Ventilators are essential to provide respiratory assistance to patients with severe COVID-19 disease who are unable to breath by themselves or need positive pressure in their lungs to overcome inflammation and fluid from infection. BilevelPAP machines are similar to ventilators in they provide a higher pressure for inspiration and a lower pressure for expiration with the difference in pressures driving breaths. There are many millions of BiLevPAP machines currently in homes around the US and the world. There are over 50,000 BilevelPAP machines in US hospital (this is similar to the about 70,000 ventilators in US hospitals at the drafting of this document.). Hundreds of thousands of BilevelPAP machines are made and sold each year. Thus, large numbers of BilevelPAP machines are readily available in homes, hospitals, and for purchase.

BilevelPAP machines are currently used in the following system configuration as shown in FIG. 3. A BilevelPAP motivator 120b blows ambient air 180 into tubing 130 pressurizing the air 184. Tubing carries pressurized air from the CPAP motivator 120b to a patient mask or other interface 140. A patient mask covers the patient nose and sometimes the mouth as well. An exhaust vent on mask allows exhaled air to be released into room. When the patient exhales, most of his exhaled air goes into the tubing and mask. This exhaled air is then exhausted into the room through the vent on the mask.

Limitations of BilevelPAP systems make their use as ventilators inadequate because that exhaled air from the patient is dispersed into the room, typically through exhaust vents on the mask, and could infect others in their vicinity (e.g. healthcare workers, family members, caregivers, other patients) and contaminate surfaces around the patient. Also, some patients require additional oxygen supplementation. Furthermore, some patients are unable to protect their airway from oral secretions and other liquids. Some patients are unable to initiate breaths on a regular basis and thus need an external trigger to initiate their breaths (i.e. if the patient does not try to breath, the machine will not automatically increase the pressure to help them breath, say 15 or some other number of times per minute).

CPAP systems are a main stay of treatment for OSA and includes a mask and blower. CPAP systems include constant pressure systems, auto-adjusting pressure systems, constant pressure with a decrease in pressure on exhalation (e.g. RESMED EPR, RESPIRONICS C-Flex), bi-level positive airway pressure systems, bi-level positive airway pressure with a backup rate, and adaptive servo controlled ventilation positive airway pressure machines. Tens of millions of CPAP masks that are gas tight and reusable are already in existence in the US. Millions of CPAP blowers are already in existence in the US.

### SUMMARY

The present invention is directed to filtered respiration that can occur according to multiple facets. The present invention includes a Continuous Positive Airway Pressure (“CPAP”) system, an adapter, and a CPAP interface. Other inventions disclosed herein relate to the modification of CPAP and BILEVEL-PAP systems into ventilators.

The CPAP system includes a gas motivator, gas channel, respiration interface, and an adapter. The gas motivator is a device that urges gas along a particular vector, and may include any form of air mover or the like. Conventional CPAP blowers are ideal in connection with the present invention. A gas channel connects to the gas motivator and leads pressurized gas to a respiration interface. The gas channel can include, conduit, piping, or any other means of conduction of a gas from one point to another. The typical gas channel includes the piping associated with conventional CPAP equipment; however, the significance of the gas channel is merely that it transports gas in the direction of a user/patient.

A respiration interface includes the delivery means of the gas from the gas motivator. Conventional forms of masks associated with CPAP systems may be utilized with the present invention, so long as they originally or as-modified meet the objectives of the present invention. The point of delivery for CPAP, and related systems, often includes a mask that covers the nose and/or mouth of a user/patient, but other forms of interface may be utilized such as internal tubing, e.g. endotracheal tube, or a laryngeal mask. The respiration interface is the point of delivery of gas to the airway of a user. The airway as discussed herein includes any respiratory channel of a user's body, including nasal, mouth, and tracheal passages. The respiration interface sealingly connects to the airway of the user, meaning that to the best of equipment's (i) intended purpose or (ii) manufacturing tolerances (an exclusive of human cooperation), there is a barrier between the airway of the user and the ambient environment in which the user is located.

The present invention includes a dedicated, filtered passage in an adapter that permits gas exhaled by a user to be cleansed of malicious agents with particular characteristics such as particulate size or chemical composition. The malicious agents of the present invention can include chemicals, viruses, bacteria, mold, or any other agent that can be harmful to those surrounding the user/patient and desirous of blocking. The adapter can be positioned on the interface, the channel, lie therebetween, or occupy any other position that it is in the path of exhaled gas. The adapter includes a respiratory outlet having a filter that obstructs an exclusive passage of exhaled gas from the user to the ambient environment. In certain versions of the present invention, the exclusive passage can be purpose-built and available originally; or in other versions of the present invention, the exclusive passage can exist by virtue of a dedicated effort to seal other passages in any component of the system that might result in exhaled air encountering the ambient environment. By “exclusive passage” it is not meant to be limiting in quantity or construction, but rather it is meant that to the extent that there is a passage from the airway to the ambient environment, it is through the one or more filters of the present invention. The “exclusive passage” can be bifurcated, or take the form of multiple branches, etc. However, the present invention results in a significant reduction in the danger posed by unfiltered exhalant spewed or aerosolized into the ambient environment by a user.

The functionality of the adapter can be integrated as a component, or a stand-alone component. For example, the adapter may be integrated, affixed, included with, or otherwise associated with an interface. The adapter may be integrated, affixed, included with, or otherwise associated with a gas channel. The adapter may be a discrete item added to the gas channel, interface, or therebetween.

These aspects of the invention are not meant to be exclusive. Furthermore, some features may apply to certain versions of the invention, but not others. Other features, aspects, and advantages of the present invention will be readily apparent to those of ordinary skill in the art when read in conjunction with the following description, and accompanying drawings.

### BRIEF DESCRIPTION OF THE DRAWINGS

FIG. 1 is a schematic view of a conventional CPAP system arrangement.

FIG. 2 is a schematic view of the system and process of the present invention.

FIG. 3 is a schematic view of a conventional BILEVEL-PAP system arrangement.

FIG. 4 is a schematic view of the system and process of the present invention.

FIG. 5 is a schematic view of the system and process of the present invention.

FIG. 6 is a schematic view of the system and process of the present invention.

FIG. 7 is a schematic view of the system and process of the present invention.

FIG. 8 is a schematic view of the user interface of the system and process of the present invention.

FIG. 9 is a schematic view of the system and process of the present invention.

### DETAILED DESCRIPTION

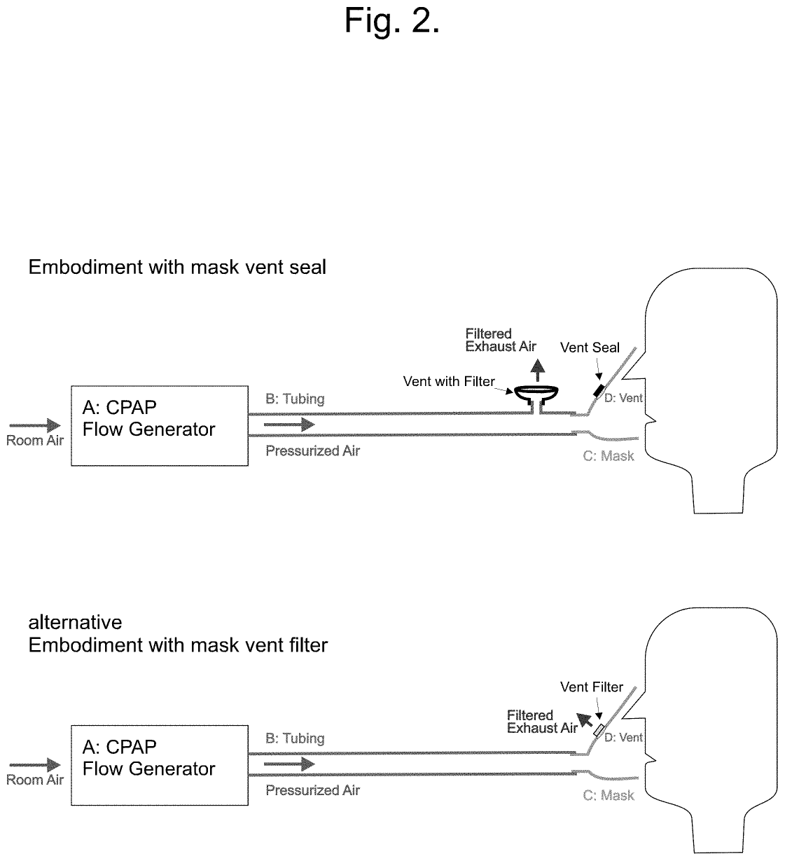

Referring first to FIG. 2, a basic embodiment of the system 100 is shown. A filter 160 may be placed over the Continuous Positive Airway Pressure (“CPAP”) mask 140 vent (e.g. by taping a filter fabric over the vent). Alternatively, a seal 149, or other impediment, may be placed over the non-filtered exhaust on the CPAP mask 140 by applying a commercial adhesive such as silicone or hot glue or caulk or other construction adhesive available at a hardware store (e.g. Home Depot). Alternatively, tape 149 or any other mechanism may also be used to seal the non-filtered exhaust on the CPAP mask.

An adapter 142 with a filtered exhaust 160 is added to the system (e.g. tubing or mask) near the patient. If the exhaust were added far from the patient it would increase the amount of “Dead Space” i.e. air the patient is ventilating (moving) that is not available for O2/CO2 exchange. The filtered exhaust prevents virus infected droplets from being dispersed into the room. The filtered exhaust may be created using the following or other methods: adding a T-shaped connector in-line with the tubing near the patient; or adding a new hole or using an existing hole (e.g. oxygen ports) on the patient mask to create a new exhaust channel; adding a filter to that new exhaust channel such as the Hudson RCI Bacterial Viral Filter model #1605. Alternatively, exhausted air may be channeled through a water seal system which includes a disinfectant.

The water seal system may be mechanically or ultrasonically agitated so that bubbles of air are broken down within the water seal chamber and exposed to the detergent in the water seal. The water seal system may then have a filter above the air above the fluid so that there is yet another level of removal of infection from droplets rising above the water system.

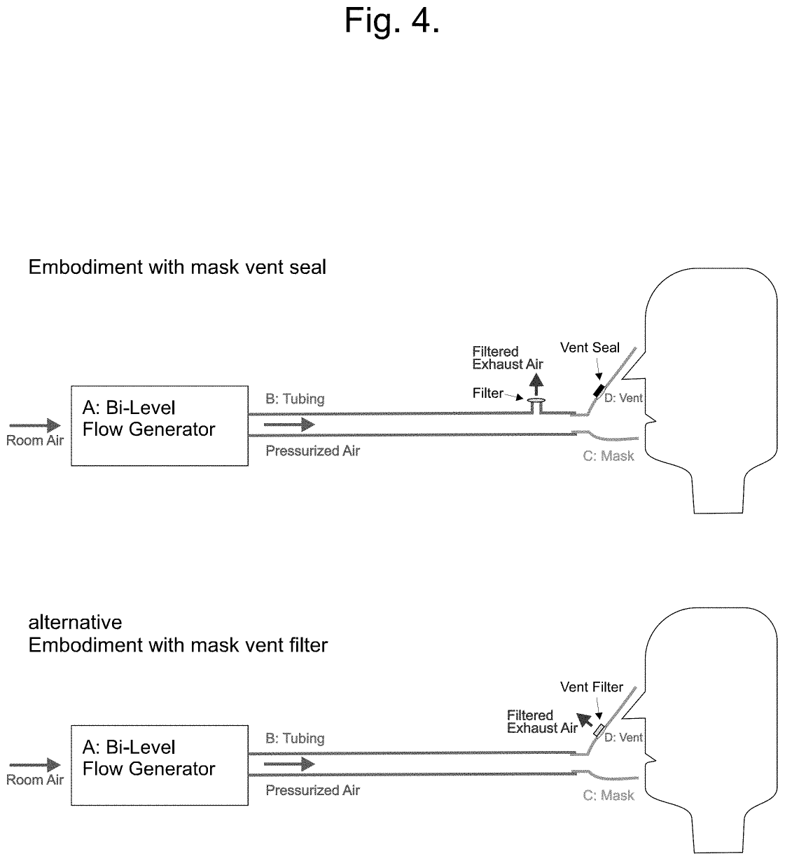

Turning now to FIG. 4, a filter 160 may be placed over the adapter outlet 146 positioned on the mask vent (e.g. by taping a filter fabric over the vent). Alternatively, a seal 149 may be placed over the non-filtered exhaust on the mask by applying a commercial adhesive such as silicone or hot glue or caulk or other construction adhesive available at a hardware store (e.g. Home Depot). Alternatively, tape 149 or any other mechanism may also be used to seal the non-filtered exhaust on the mask.

An exhaust 146 with a filter 160 is added to the system (e.g. tubing or mask) as an adapter 142 near the patient. If the exhaust were added far from the patient it would increase the amount of “Dead Space” i.e. air the patient is ventilating (moving) that is not available for O2/CO2 exchange. The filtered exhaust prevents virus infected droplets from being dispersed into the room. The filtered exhaust may be created using the following or other methods: adding a T-shaped connector in-line with the tubing near the patient; or adding a new hole or using an existing hole (e.g. oxygen ports) on the patient mask to create a new exhaust channel; adding a filter to that new exhaust channel such as the Hudson RCI Bacterial Viral Filter model #1605.

Alternatively, exhausted air may be channeled through a water seal system which includes a disinfectant. The water seal system may be mechanically or ultrasonically agitated so that bubbles of air are broken down within the water seal chamber and exposed to the detergent in the water seal. The water seal system may then have a filter above the air above the fluid so that there is yet another level of removal of infection from droplets rising above the water system.

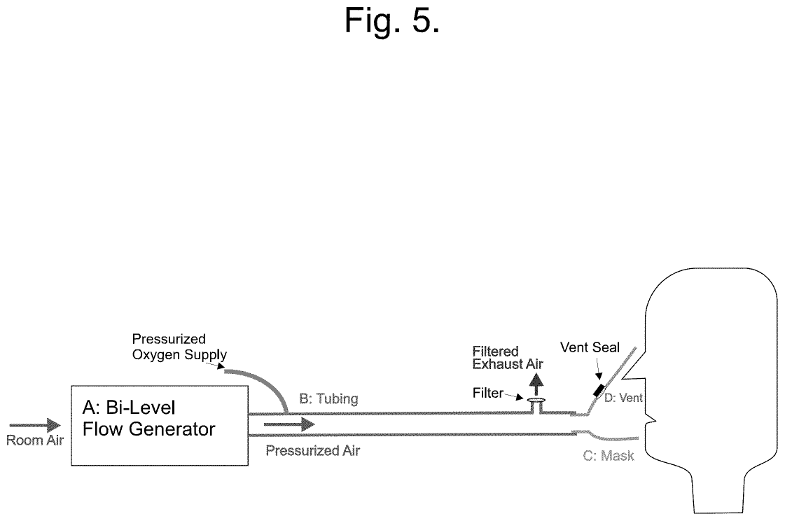

Turning now to FIG. 5, one may connect a pressurized oxygen supply, as available from a portable oxygen concentrator 150 or central supply in most hospitals, to the tubing 152 that leads to the channel 130.

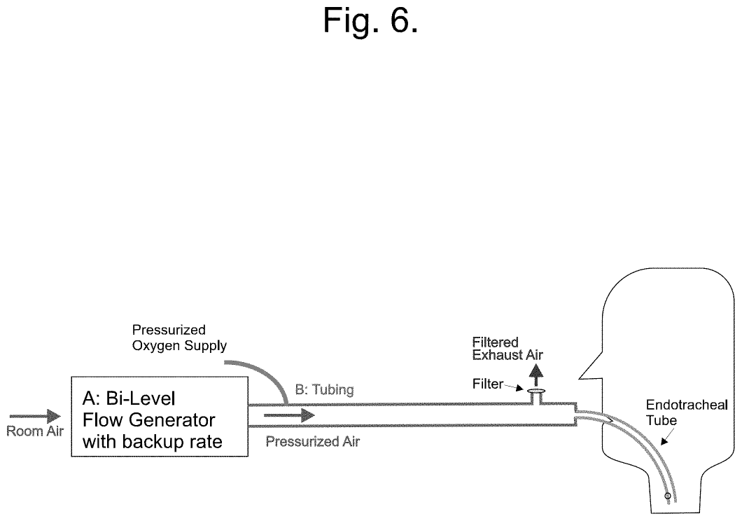

Turning now to FIG. 6, an airway protection device as a user interface 140 that precludes oral secretions or other materials from the oropharynx or nasopharynx from entering the trachea is placed between the channel 130 and the user's trachea. Connecting the channel from the bi-level PAP machine to an endotracheal tube or a laryngeal mask or a tracheostomy tube or other portal may aid in ventilating users' lungs more directly.

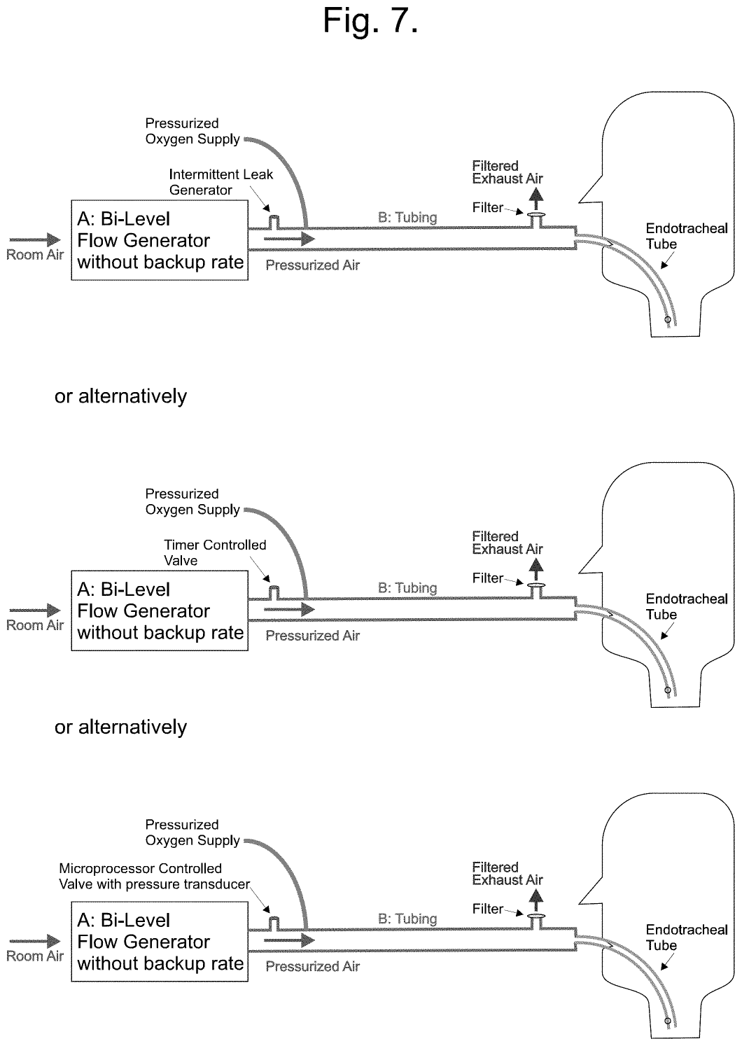

Turning now to FIG. 7, while some Bi-Level PAP machines may be programmed to have a “backup rate” whereby they initiate inspiration after a predefined number of seconds if the patient has not initiated inspiration spontaneously, the vast majority of bi-level PAP machines that have been sold over the last decade in the US or the world do not have this functionality. However, ordinary bi-level PAP machines can be triggered to initiate an inspiration by mimicking the subtle decrease in pressure that the machine normally senses when a patient spontaneously initiates a breath.

This subtle decrease in pressure can be mimicked by introducing a very brief (e.g. 100 msec) leak in the tubing circuit near the machine. If the leak is introduced near the motivator 120b, then the tube will not have any infected air that the patient had exhaled. To increase safety, a filter may 160 be placed around the area where the leak is created. One may place a T-shaped connector in-line with the tubing 130 near the machine 120b. One may place a valve 134 on the part of the “T” that is not in-line with the tubing (e.g. a solenoid valve or a rotating disc valve or other type of valve). One may place a controller 170 in communication with the valve 134 that opens the valve (e.g. for 100 ms to 2000 ms) at regular intervals (e.g. every 4 seconds) so that the bi-level PAP machine senses a decrease in pressure that triggers the bi-level PAP machine to initiate a breath. The controller may be built using a digital microprocessor controller or a simple timer chip such as the 555 timer chip introduced by SIGNETICS in 1972 or a mechanical timer.

Alternatively, a mechanical timer such as that used in a pulsating shower head may be used to intermittently allow and obstruct the leak (see e.g., U.S. Pat. No. 4,254,914, the contents of which are hereby incorporated by reference). Alternatively, a pressure transducer (e.g. piezoelectric crystal) operatively connected to a microcomputer 170 may be used to determine if the patient has spontaneously triggered a breath within the last four seconds (or some other time) and if not, then trigger a breath by opening the solenoid valve. The timer controlled valve may be powered by a battery or by splitting the power supply to the bi-level PAP machine. The microcontroller may be powered by a battery or by splitting the power supply to the bi-level PAP machine. So that a full breath is delivered when the valve opens, a Tₘᵢₙ minimum (minimum inspiratory time) and a Tₘₐₓ maximum will need to be set on the bilevel flow generator (e.g. two seconds of minimum inspiratory time and two seconds of maximum inspiratory time).

Conventional Bilevel CPAP systems without a backup respiratory rate are unable to initiate respirations for patients. A backup respiratory rate is particular essential for patients who have been sedated to allow placement of a Protected Airway interface 140 or who are so unwell with an infection that their brain's respiratory control centers are unable to regularly initiate breaths. One could intermittently introduce a leak in the CPAP tube which will in-turn intermittently lower the pressure in the CPAP tube and thus cause the bilevel CPAP machine to interpret that the patient has initiated an inspiration which will in-turn cause the bilevel CPAP machine to increase its pressure to the inspiratory pressure (from the expiratory pressure). The bilevel CPAP machine may have a fixed inspiratory time set so that a consistent full breath is delivered. This is achieved by specifying the minimum and maximum inspiratory times for breaths (e.g. Ti minimum and Ti maximum may both be set to two seconds).

The intermittent leak is created by an air powered oscillating machine which intermittently covers and uncovers a leak from the CPAP system. The oscillating machine may be built by using a crank link slider mechanism. The crank link slider mechanism may be powered by an air stream from the CPAP machine. The airstream may cause a fan-like element, e.g. a turbine, to rotate. The fan like element's rotation may drive a link and slider to oscillate back and forth. The slider shall intermittently cover and uncover a leak from the CPAP circuit. The rate of said oscillation may be adjusted by the amount of the air stream which creates the rotary motion. The air stream may be adjusted by a simple ball valve. Thus, adjustment of the ball valve can adjust the rate at which the CPAP circuit intermittently experiences a leak and thus the rate at which the bilevel CPAP machine initiates an inhalation.

In more detail, CPAP tubes are generally 19 mm in inside diameter and at baseline when treating patients with ARDS (acute respiratory distress syndrome) have a pressure of approximately 10 cm H2O and a flow rate of approximately 20 liters per minute. In a tube a leak (e.g. 2 liters per minute) that can be introduced for a short period of time (e.g. 100 milliseconds) at regular intervals (e.g. every 4 seconds). The time between the intermittent leaks being introduced can be adjustable by adjusting a valve that regulated the air to the below air-powered machine. The intermittent leak can be produced by an air powered machine. The air powered machine shall be powered by diverting a small part of the air flow in the tube to a rotary engine that then uses a crank-link-slider mechanism to intermittently cover and uncover a leak area that is effectively connected to the air in the CPAP tube. To increase the reliability of this system, two or more air of the air powered machines may be synchronized and placed in parallel or series on the CPAP tube.

Alternatively, this may be done is by attaching T-shaped connector in-line with the tubing 130 near the machine. Place a valve on the part of the “T” that is not in-line with the tubing (e.g. a solenoid valve or a rotating disc valve or other type of valve). Place a controller on the valve that opens the valve (e.g. for 100 ms) at regular intervals (e.g. every 4 seconds) so that the bi-level PAP machine senses a decrease in pressure that triggers the bi-level PAP machine to initiate a breath by increasing its pressure. The controller may be built using a digital microprocessor. Alternatively the controller may use a timer chip such as the 555 chip introduced by SIGNETICS in 1972

Alternatively the controller may use a mechanical timer. Examples of mechanical timers can include a piston in a cylinder moved at a constant rate with a slit in the channel in which the piston is moving so that intermittently a leak is generated. Another example of a mechanical timer is a motor that rotates at a constant speed and moves a disc that intermittently exposes a leak. A pressure transducer (e.g. piezoelectric crystal) may be operatively connected to a microcomputer and be used to determine if the patient has spontaneously triggered a breath within the last four seconds (or some other number of seconds) and if not, then trigger a breath by opening the solenoid valve. The valve may be powered by an independent power supply, a battery, or by splitting the power supply to the bi-level PAP machine. The microcontroller may be powered by an independent power supply, a battery, or by splitting the power supply to the bi-level PAP machine. So that a full breath is delivered when the valve opens, an inspiratory time may be specified (on some Bilevel CPAP machines this is done by specifying a minimum and a maximum inspiratory time e.g. two seconds).

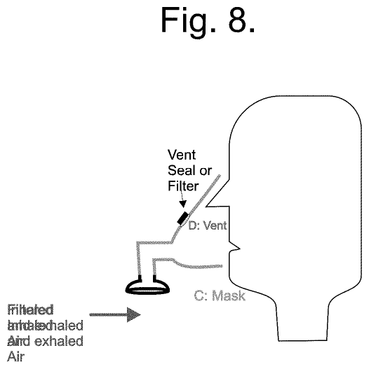

Turning now to FIG. 8, CPAP masks may function as reusable personal protective equipment. CPAP masks in their current state are inadequate for use as personal protective equipment when working with patients with infections such as COVID-19 since they do not filter incoming air. CPAP masks in their current state are inadequate for use by patients who have an infection such as COVID-19 since they do not filter exhaled air. CPAP masks are uncomfortable to use as personal protective equipment since due to their gas tight nature, they cause heat and sweat buildup.

CPAP masks, and other interfaces, may function as reusable personal protective equipment under the present invention. The filter may be connected to the port to which CPAP tubing normally connects. Alternatively, the filter may be connected to a new or existing port on the mask provided that other ports to the mask are sealed (e.g. by plumbing pipe plugs, tape, silicone, or other sealant or other material). The filter should have a filter medium capable of blocking the particles to be filtered (e.g. more than 95% of particles larger than 0.3 microns). The filter may have a surface area that allow inhalation without excessive respiratory effort (e.g. 200 cm², which is much larger than the cross sectional area of conventional CPAP tubing). The filter could be a prefabricated housing with a filter fabric (e.g. HUDSON RCI Bacterial Viral Filter model #1605) Alternatively, such filter may be made of a pouch of filter fabric and connected securely to the end of the CPAP tubing port (e.g. by using a stainless steel hose clamp). Alternatively, such filter could be made of a rigid structure with an overlaying filter fabric. The rigid structure may be 3-D printed or made from standard plumbing piping fittings (e.g. ¾ inch copper fittings). If the plumbing piping fittings are not available in an ideal size, tape may be wrapped around either the end of the piping or around the mask's CPAP tubing connector so that an adequate seal is formed. Alternatively, the filter may be made from an alternate means as from which filters may be made.

The pipe fitting may be rigid or flexible. The filter may be created by using available tubing with an diameter that may fit the diameter of the CPAP tubing connector (e.g. 22 mm). The tubing may either go over the mask's tubing connector or inside the mask's tubing connector. The filter may be created by using ⅞ inch plumbing piping that may be made from PVC or copper or other material. The surface area of the filter may be increased by using a piece of filter fabric that is wrapped over the cpap tubing connector and secured by any available means such as a stainless steel hose clamp. The filter may be created from a piece of high efficiency air filter material (e.g. material rated as MERV 13 or MPR 2200 or FPR 10/Black) used in HVAC system air filters.

Another prudent measure is to seal an exhale vent on a mask or alternatively place a filter over the vent. The seal may be made by any means available including without limitation with nonporous tape (e.g. plastic tape such as packing or scotch tape) or a sealant (e.g. silicone, caulk, glue). The seal may be made from inside, outside, or both sides of the mask. Alternatively, one may adhere a filter capable of blocking infection particles and carrier particles over exhaust vent on mask.

Another prudent measure is to adhere filter over or seal backup inhalation port on mask, if one is present. A filter capable of blocking infection particles and carrier particles may be adhered over any backup inhalation port on mask. Alternatively, one may seal any backup inhalation port on mask. The seal may be made by any means available including without limitation with nonporous tape (e.g. plastic tape such as packing or scotch tape) or a sealant (e.g. silicone, caulk, glue). The seal may be made from inside, outside, or both sides of the mask. If the auxiliary inhalation port on mask has a flap that can cover it, the flap may be taped so that the auxiliary port remains in the closed position in addition to or instead of sealing the auxiliary inhalation port on mask from outside. An emergency removal flap of tape (e.g. by the tape being folded over onto itself at an edge) or other method may be left over the auxiliary inhalation port on mask in the event it needs to emergently be removed.

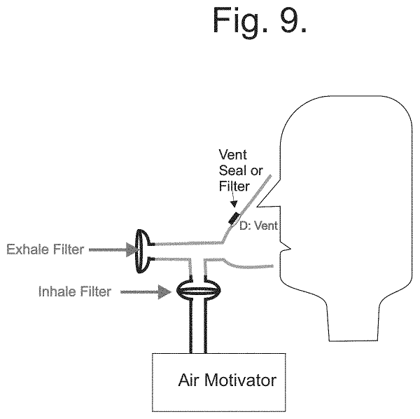

Turning now to FIG. 9, a quiescent flow blower may be utilized. A blower may be added before the air intake filter of the mask so that there is a steady stream of air going through the mask. This air stream may pressurize the mask to avoid contaminants entering the mask from lapses in the masks seal with the patient's face. The air stream will also avoid perspiration and facilitate breathing. The blower may be battery powered or powered by an external power supply. The blower may produce flows as low as ten milliliters per minute or any higher flow. Flows of 20 liters per minute will provide most of the air a typical adult requires at rest. Higher flows of 40 liters per minute will accommodate light activity. If flows less than 6 liters per minute are used then the blower would ideally channel air within the mask so that air blown in would preferentially be inhaled (e.g. by directing the inflow of the blower towards wearers nares such as by use of existing oxygen ports on many masks). A separate exhalation channel and filter should also be added to allow a lower resistance path to exhausting exhaled air.

The blower may provide flows sufficient to maintain a positive pressure in the patient mask. Said positive pressure may small, for example 0.5 cm H2O.

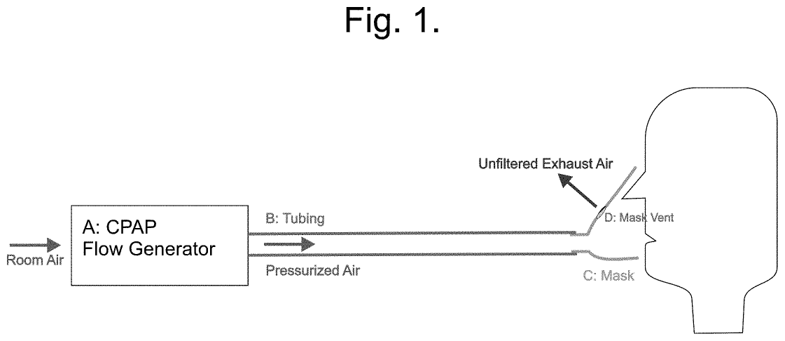

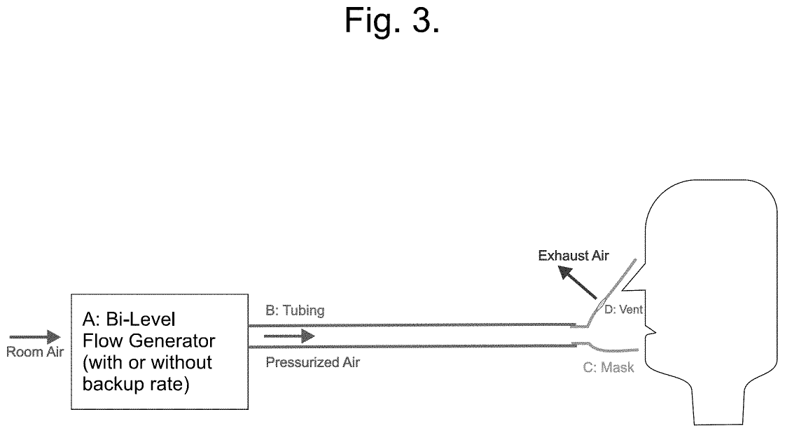

Returning to FIGS. 1-7, modern CPAP systems contain a blower, flow sensor, pressure sensor, microcontroller, cellular modem, and software. These are the same elements needed whether the system serves as a simple fixed pressure CPAP as was sold in the 1980s or a state of the art adaptive servo-controlled ventilator with bi-level functionality and a backup rate. There is no simple method to modify the units currently sold for use as simple CPAP machines so that they can serve as more advances systems such as bilevel cpap machines or ventilators. Such functionality would help individual patients treating their sleep apnea because their machines would be able to be switched from CPAP to a higher level of function. Such functionality would help at times of shortage of ventilators as has become the case during the COVID-10 pandemic.

CPAP machines sold in the 1980s were built to last decades whereas those sold presently often fail after a few years. A cynic might conclude that this perhaps planned obsolescence was to drive recurrent revenues for manufacturers. Current CPAP masks do not have a mechanism whereby exhaled air may be filtered to prevent transmission of infection to other individuals or surfaces in their vicinity.

Vents on CPAP masks do not have a method to change their size or resistance based on the CPAP pressure the patient is using. Vents on CPAP masks are designed to leak air so that exhaled air is not rebreathed. The amount of air that a particular patient needs to exhaust is largely independent of the CPAP pressure the patient requires to maintain their airway when treating their sleep apnea. For example, if a patient's minute ventilation is 5 LPM, they may need to exhaust 20 LPM through their mask to have a safe level of rebreathing. A CPAP mask that exhausts 20 LPM at a low CPAP pressure of 4 cm H2O would leak about 60 LPM, or three times as much air as needed, at a CPAP pressure of 12 cm H2O. Leaked air causes noise and mechanical disturbance both to patients and bed partners.

Current CPAP systems do not include a capnometer to measure patient or expired CO2 levels or the level of CO2 being inhaled. Current CPAP systems do not include an oximeter to measure either patient O2 levels or expired O2 levels. Current CPAP systems do not include an accelerometer that could provide guidance on patient's arousals. Therefore, a CPAP system that through its software may be converted to any of these modes with the entry of a license code either on the unit or by transmission of a software license directly through the internet or via a cell phone or other trackable means may be a desirable solution. CPAP machines could be sold with software licenses that were renewable and only allowed for function for a particular duration of time. Thus providing a mechanism for recurrent revenues for manufacturers without crippling patients in the event of premature device failure and without environmental pollution from unnecessary manufacture. The codes or licenses enabling additional functionality or extending the duration of function could be provided based on payment of a license fee over the interne or otherwise. A CPAP mask with a slot at the exhalation vent whereby a filter or seal may be inserted to filter or block exhaled air. The slot could be a square area similar to a picture frame that covers the vent with edges on three out of four sides where through the fourth side the filter or seal could be slid into place. The filter or seal that is slid into place could vary in resistance or in the fraction of its area that is a seal vs a filter depending on the patient's ventilator needs and the pressure the patient is using so that the exhaust flow rate is appropriate for the patient and his pressure requirements.

It is a feature of the present invention that CPAP machines with an operationally connected capnometer located in the mask or that could be worn on the patient's skin to assess adequacy of ventilation and give guidance to the patient and/or their physician on adequacy of therapy. It is a further feature that CPAP machines with an operationally connected oximeter located in the mask or that could be worn on the patient's skin to assess adequacy of ventilation and give guidance to the patient and/or their physician on adequacy of therapy. It is a further feature that CPAP machines with an operationally connected accelerometer in the mask or that could be worn on the patient's skin to assess for arousals and give guidance to the patient and/or their physician on adequacy of therapy. Aforementioned guidance could be directly on the machine, via a software application to a patient's portable computing device connected via BLUETOOTH or local area network protocol or other means, or via the Internet or other wired or wireless means. Aforementioned guidance could be provided via an expert system and/or telemedicine.

Exemplary traits of the present invention include:
Facially sealed airtight mask system that includes an interface that covers the nose, mouth, or both, a tube or other operative connector, and filter media (FIG. 8)
Said airtight mask may be transparent
Said filter media may be connected by a tube
Said tube may be in 2 parts
Said tube(s) may go up the cheeks to the top of the head
Said tube(s) may wrap around the ears and then go towards the front of user's neck and then towards the users' chest
Said tube(s) may wrap around the ears and then go towards the users' chest
Said tube(s) may wrap around the ears and then go towards the users' back
Said tube may go from the mask to the user's chest
Said tube may go from the mask to the user's back
Said tube may go from the mask to the user's arms
Said tubes connects to filter media
Said filter media shall be sealed so that its only exits and entries are through the filter media or through the said tube connections
said filter media may be shaped so that it has a large surface area (e.g. >200 cm\hat{ }2) and small volume (e.g. <10 cc)
said filter media may be in the shape of an envelope with a single point of entry and exit of air into the inside of the filter media space
said filter media shall be sealed so that only desired matter can traverse the filter media (e.g. o2, co2)
A Wearable CPAP machine (FIG. 9)
Said CPAP machine having an inbound air filter
Said CPAP machine having an outbound air filter
Said CPAP machine being battery powered
Said CPAP machine measuring flow into mask
Said measuring system may be mechanical
   Said mechanical flow meter may be based on a tapered chamber
   Said mechanical flow may be based on a rotary meter
   Said mechanical flow may be based on a fan like device
Said CPAP machine measuring flow out of mask
Said CPAP machine measuring pressure at mask (after inbound filter)
Said CPAP machine recognizing breaths taken by user
Said CPAP machine having leak alarm
Said alarm may be based on constant flow
Said CPAP machine having low flow warning or alarm
Said low flow warning may be a mechanical visual indicator
Said low flow warning may be an auditory alarm
Said CPAP machine having low pressure warning or alarm system
Pressure sensor between HEPA filter and patient interface so that pressure drop across HEPA filter accounted for in assessing pressure delivered to patient
Filtered Vent
Flow indicator to provide warning if insufficient flow (e.g. due to filter clogging)
Vent from bilevel with intermittent leak from microcontroller or fluidic oscillator
Vent from CPAP with PEEP and switching between PEEP and CPAP by microcontroller or fluidic oscillator

Although the present invention has been described in considerable detail with reference to certain preferred versions thereof, other versions would be readily apparent to those of ordinary skill in the art. Therefore, the spirit and scope of the appended claims should not be limited to the description of the preferred versions contained herein.

### INDUSTRIAL APPLICABILITY

A novel positive airway pressure personal protective equipment system.

A novel method of reducing infection spread when a CPAP machine is used by a patient with an infection. This is especially important in nursing homes, hospitals, recovery rooms, emergency rooms and ICU settings to avoid infecting other healthcare workers or patients or surfaces.

A novel method of reducing infection spread when a BilevelPAP machine is used by a patient with an infection. This is especially important in nursing homes, hospitals, recovery rooms, emergency rooms and ICU settings to avoid infecting other healthcare workers or patients or surfaces.

A novel method of reducing infection spread when a BilevelPAP machine is used as a ventilator. This is especially important in nursing homes, hospitals, recovery rooms, emergency rooms and ICU settings to avoid infecting other healthcare workers or patients or surfaces.

A novel method of providing supplemental oxygen to patients using a bi-level PAP device converted to a ventilator.

A novel method of causing a BilevelPAP machine without a backup respiratory rate to have a backup respiratory rate.

What is claimed is:

1. A Continuous Positive Airway Pressure system for a user in an ambient environment, said machine comprising:
a gas motivator;
a gas channel, affixed to said gas motivator, to accept and conduct pressurized gas; and
a respiration interface, affixed to said gas channel both to accept pressurized gas and accept exhaled gas, having a housing dimensioned to envelope an airway of the user;
and
an adapter comprising respiratory outlet having a filter obstructing an exclusive passage of said exhaled gas from the user to the ambient environment.

2. The system of claim 1 wherein said housing is transparent.

3. The system of claim 1 wherein said adapter includes said filter within a flexible tube.

4. The system of claim 1 wherein said tube includes multiple tube divisions.

5. The system of claim 4 wherein said tube divisions are dimensioned to abuttingly flank a face of the user with an upwardly-facing exhaust aperture.

6. The system of claim 4 wherein said tube divisions are dimensioned to descend below a neckline of the user.

7. The system of claim 4 wherein said tube divisions are dimensioned to ventrally descend below a neckline of the user.

8. The system of claim 4 wherein said tube divisions are dimensioned to dorsally descend below a neckline of the user.

9. A Wearable Continuous Positive Airway Pressure (“CPAP”) interface for a user in an ambient environment, said interface comprising:
an interface body, adapted to accept pressurized gas from a CPAP gas motivator, dimensioned to sealingly connect to an airway of the user;
an adapter with a respiratory outlet having a filter obstructing an exclusive passage of exhaled gas from the user to the ambient environment;
retention means, affixed to said interface body, for the positioning of said interface body adjacent to the airway of the user.

11. The interface of claim 10 wherein said interface includes an inbound air filter.

12. The interface of claim 10 wherein said interface includes an outbound air filter.

13. A Wearable Continuous Positive Airway Pressure system for a user in an ambient environment, said machine comprising:
a respiration interface, to accept pressurized gas and accept exhaled gas, dimensioned to envelope an airway of the user;
an adapter comprising respiratory outlet having a filter obstructing an exclusive passage of said exhaled gas from the user to the ambient environment;
a gas motivator, supported by said respiration interface; and
a gas channel, affixed to said gas motivator, to accept and conduct pressurized gas between said gas motivator and said respiration interface.

14. The system of claim 13 wherein said gas channel includes an inlet channel and an exhaust channel.

15. The system of claim 14 wherein said inlet channel includes an inlet filter.

16. The system of claim further comprising a flow meter in fluid communication with said inlet channel.

17. The system of claim 13 further comprising a power source in electrical communication with said gas motivator.

18. The system of claim 17 wherein said power source is supported by said gas motivator.
